(19) United States
(12) Patent Application Publication
(10) Pub. No.: US 2020/0392485 A1
(43) Pub. Date: Dec. 17, 2020

(54) COMPOSITIONS AND METHODS FOR IMPROVED cDNA SYNTHESIS

(71) Applicant: Pacific Biosciences of California, Inc., Menlo Park, CA (US)

(72) Inventors: Brendan Galvin, Menlo Park, CA (US); Heather Ferrao, San Mateo, CA (US)

(21) Appl. No.: 16/870,444

(22) Filed: May 8, 2020

Related U.S. Application Data
(60) Provisional application No. 62/845,609, filed on May 9, 2019.

(51) Int. Cl. C12N 15/10; C12Q 1/6853

(52) U.S. Cl. CPC C12N 15/1096; C12Q 1/6853

(57) ABSTRACT

Modified template switching oligonucleotides (TSOs), compositions containing modified TSOs, and methods for employing modified TSOs to synthesize cDNA from RNA templates, where the cDNA includes an adapter region at the 3′ end, are provided. The modified TSOs include at least one 2′-fluoro-ribonucleotide in the 3′ annealing region and provide for improved conversion of RNA into full-length cDNA, resulting in increased yield and complexity as compared to non-modified TSOs and thereby finding use in generating cDNA from samples having low RNA input.

### CROSS-REFERENCE TO RELATED APPLICATIONS

This application claims priority to and the benefit of provisional patent application U.S. Ser. No. 62/845,609, filed May 9, 2019, entitled “COMPOSITIONS AND METHODS FOR IMPROVED cDNA SYNTHESIS” by Brendan Galvin and Heather Ferrao, which is incorporated herein by reference in its entirety for all purposes.

### INCORPORATION-BY-REFERENCE OF MATERIAL SUBMITTED BY U.S.P.T.O. eFS-WEB

The instant application contains a Sequence Listing which is being submitted in computer readable form via the United States Patent and Trademark Office eFS-WEB system and which is hereby incorporated by reference in its entirety for all purposes. The txt file submitted herewith contains a 2 KB file (Ser. No. 01/022,401_2020-09-04_SequenceListing.txt).

### BACKGROUND OF THE INVENTION

The production of full-length cDNAs from RNA templates is important for a wide variety of genetic analyses, including characterizing gene structure and function. Many cDNA library construction methods are not designed to generate full-length cDNA products, often missing the 5′ ends of the RNA template. As a result, full-length RNA templates are significantly underrepresented in many cDNA libraries. One approach to improving the representation of full-length mRNAs in cDNA libraries is through the use of template switching oligonucleotides (TSOs) which act as a synthetic template region located adjacent to the 5′ terminus of the original RNA template, thereby allowing for the addition of adapter regions at the 3′ end of cDNA templates. These adapter regions can be exploited in downstream processes to preferentially analyze full-length cDNA species, e.g., amplification, adapter ligation, and sequencing.

While the use of TSOs has been beneficial in full-length cDNA/cDNA library production, limitations remain. For example, there is a need for improving the yield of full-length cDNA production from RNA samples with very low/limited input levels. Aspects of the present disclosure address this, and other, needs.

### SUMMARY OF THE INVENTION

The present disclosure provides modified template switching oligonucleotides (TSOs), compositions containing modified TSOs, and methods for employing modified TSOs to synthesize cDNA from RNA templates, where the cDNA includes an adapter region at the 3′ end. The modified TSOs include at least one 2′-fluoro-ribonucleotide in the 3′ annealing region and provide for improved conversion of RNA into full-length cDNA resulting in increased yield and complexity as compared to non-modified TSOs, thereby finding use in generating cDNA from samples having low RNA input.

Aspects of the present disclosure include a method for generating a complementary DNA (cDNA) strand with a 3′ adapter region, the method comprising: combining an RNA template with a cDNA synthesis primer (sometimes referred to as a reverse transcription (RT) primer), a template switching oligonucleotide (TSO), and a reverse transcriptase under cDNA synthesis conditions, wherein the TSO comprises a 5′ adapter region and a 3′ annealing region comprising at least one 2′-fluoro-ribonucleotide, wherein (i) the cDNA synthesis primer anneals to the RNA template and the reverse transcriptase generates an RNA-cDNA intermediate from the annealed cDNA synthesis primer, wherein the cDNA strand of the RNA-cDNA intermediate comprises a 3′ overhang; and (ii) the 3′ annealing region of the TSO anneals to the 3′ overhang of the RNA-cDNA intermediate and the reverse transcriptase extends the 3′ end of the cDNA strand of the RNA-cDNA intermediate using the annealed TSO as a template; thereby generating a cDNA strand comprising a 3′ adapter region.

In certain embodiments, the 3′ annealing region comprises three ribonucleotide residues. In certain embodiments, the 3′ annealing region comprises one 2′-fluoro-ribonucleotide. In certain embodiments, the 3′ annealing region comprises two 2′-fluoro-ribonucleotides. In certain embodiments, the 3′ annealing region comprises three 2′-fluoro-ribonucleotides.

In certain embodiments, at least one 2′-fluoro-ribonucleotide is 2′-fluoro-riboguanine (2′fG). In certain embodiments, any non-2′fG ribonucleotides in the 3′ annealing region of the TSO are riboguanine (rG) ribonucleotides. In certain embodiments, the 3′ annealing region comprises a universal nucleotide base. In certain embodiments, the universal nucleotide base is selected from the group consisting of riboinosine (rI) and 5′ 5-nitroindole (5′NI). In certain embodiments, the 3′ annealing region comprises a degenerate ribonucleotide base (rN).

In certain embodiments, the 3′ annealing region of the TSO, in a 5′ to 3′ direction, is selected from the group consisting of: rG-rG-2′fG; rG-2′fG-2′fG; 2′fG-2′fG-2′fG; 2′fG-2′fG-2′fG-2′fG; rN-2′fG-2′fG; rI-2′fG-2′fG; and 5′NI-2′fG-2′fG. In certain embodiments, the 3′ annealing region of the TSO, in a 5′ to 3′ direction, is: rG-2′fG-2′fG. In certain embodiments, the 3′ annealing region of the TSO, in a 5′ to 3′ direction, is: 2′fG-2′fG-2′fG.

In certain embodiments, the method further comprises amplifying the cDNA strand comprising the 3′ adapter region.

In certain embodiments, the 5′ adapter region of the TSO further comprises one or more of: a barcode sequence, a unique molecule identifier (UMI), an amplification primer sequence, a sequencing primer sequence, a capture primer sequence, a sequence-specific nuclease cleavage site, a modified nucleotide, a biotinylated nucleotide, and a 5′ modification.

In certain embodiments, the cDNA synthesis primer comprises a second 5′ adapter region and a 3′ RNA annealing region.

In certain embodiments, the second 5′ adapter region of the cDNA synthesis primer comprises one or more of: a barcode sequence, a unique molecule identifier (UMI), an amplification primer sequence, a sequencing primer sequence, a capture primer sequence, a sequence-specific nuclease cleavage site, a modified nucleotide, a biotinylated nucleotide, and a 5′ modification.

In certain embodiments, the 5′ adapter region of the TSO comprises a first amplification primer sequence and the second 5′ adapter region of the cDNA synthesis primer comprises a second amplification primer sequence, the method further comprising performing a PCR on the cDNA using a primer pair specific for the first and second amplification primer sequences. In certain embodiments, the first and second amplification primer sequences are the same. In certain embodiments, the first and second amplification primer sequences are different.

In certain embodiments, the RNA template is selected from the group consisting of: mRNA, non-coding RNA including miRNA, siRNA, piRNA, lncRNA, and ribosomal RNA. In certain embodiments, the RNA template is an mRNA. In certain embodiments, the mRNA has a 7-methylguanosine CAP structure attached at the 5′-end. In certain embodiments, the mRNA template has a poly-A tail at the 3′-end.

In certain embodiments, the cDNA synthesis primer comprises a 3′ poly-T sequence complementary to the poly-A tail. In certain embodiments, the cDNA synthesis primer comprises a 3′ sequence complementary to at least one target RNA.

In certain embodiments, the cDNA synthesis primer and the TSO are combined with the RNA template simultaneously.

In certain embodiments, the cDNA synthesis primer and the reverse transcriptase are combined with the RNA template under cDNA synthesis conditions to form a pre-extension mixture to generate the RNA-cDNA intermediate prior to combining with the TSO.

In certain embodiments, the pre-extension mixture is incubated from 10 minutes to 4 hours prior to combining with the TSO. In certain embodiments, the pre-extension mixture is incubated from 30 minutes to 2 hours prior to combining with the TSO. In certain embodiments, the pre-extension mixture is incubated for about 1 hour prior to combining with the TSO.

Aspects of the present disclosure include a method for generating adapter-containing cDNAs from a sample comprising mRNAs, the method comprising: (a) obtaining a sample comprising mRNAs having 3′ poly-A tails; (b) producing a cDNA synthesis reaction by contacting the sample with a cDNA synthesis primer and a reverse transcriptase under cDNA synthesis conditions, wherein the cDNA synthesis primer comprises a 3′ poly-T annealing region and the reverse transcriptase adds 3′ terminal nucleotide overhangs to the 3′ ends of cDNAs; (c) allowing the cDNA synthesis reaction to proceed for from 10 minutes to 4 hours to produce cDNAs with 3′ overhangs; (d) adding a template switching oligonucleotide (TSO) to the cDNA synthesis reaction, wherein the TSO comprises a 5′ adapter region and a 3′ annealing region comprising three ribonucleotides, wherein at least one of the ribonucleotides is a 2′-fluoro-riboguanine (2′fG) nucleotide; and (e) incubating the cDNA synthesis reaction under conditions that allow the 3′ annealing region of the TSO to anneal to the 3′ overhangs of the cDNAs and that allow extension of the 3′ end of the cDNAs using the annealed TSO as a template, thereby generating adapter-containing cDNAs.

In certain embodiments, the 3′ annealing region comprises one 2′fG nucleotide. In certain embodiments, the 3′ annealing region comprises two 2′fG nucleotides. In certain embodiments, the 3′ annealing region comprises three 2′fG nucleotides. In certain embodiments, any non-2′fG nucleotides in the 3′ annealing region of the TSO are riboguanine (rG) nucleotides. In certain embodiments, the 3′ annealing region of the TSO, in a 5′ to 3′ direction, is selected from the group consisting of: rG-rG-2′fG; rG-2′fG-2′fG; 2′fG-2′fG-2′fG; 2′fG-2′fG-2′fG-2′fG; rN-2′fG-2′fG; rI-2′fG-2′fG; and 5′NI-2′fG-2′fG. In certain embodiments, the 3′ annealing region of the TSO, in a 5′ to 3′ direction, is: rG-2′fG-2′fG. In certain embodiments, the 3′ annealing region of the TSO, in a 5′ to 3′ direction, is: 2′fG-2′fG-2′fG.

In certain embodiments, the 5′ adapter region of the TSO further comprises one or more of: a barcode sequence, a unique molecule identifier (UMI), an amplification primer sequence, a sequencing primer sequence, a capture primer sequence, a sequence-specific nuclease cleavage site, a modified nucleotide, a biotinylated nucleotide, and a 5′ modification.

In certain embodiments, the cDNA synthesis primer comprises a second 5′ adapter region and a 3′ RNA annealing region. In certain embodiments, the second 5′ adapter region of the cDNA synthesis primer comprises one or more of: a barcode sequence, a unique molecule identifier (UMI), an amplification primer sequence, a sequencing primer sequence, a capture primer sequence, a sequence-specific nuclease cleavage site, a modified nucleotide, a biotinylated nucleotide, and a 5′ modification.

In certain embodiments, the 5′ adapter region of the TSO comprises a first amplification primer sequence and the second 5′ adapter region of the cDNA synthesis primer comprises a second amplification primer sequence, the method further comprising performing a PCR on the cDNA generated in step (e) using a primer pair specific for the first and second amplification primer sequences.

In certain embodiments, step (c) is allowed to proceed for from 30 minutes to 2 hours. In certain embodiments, step (c) is allowed to proceed for about 1 hour.

Aspects of the present disclosure include a template switching oligonucleotide (TSO) comprising a 5′ adapter region and a 3′ annealing region, wherein the 3′ annealing region is configured to anneal to a 3′ overhang of a cDNA strand of an RNA-cDNA intermediate, wherein the TSO is capable of serving as a template for extension of the 3′ end of the cDNA strand, and wherein the 3′ annealing region comprises at least one 2′-fluoro-ribonucleotide.

In certain embodiments, the 3′ annealing region comprises three ribonucleotide residues. In certain embodiments, the 3′ annealing region comprises one 2′-fluoro-ribonucleotide. In certain embodiments, the 3′ annealing region comprises two 2′-fluoro-ribonucleotides. In certain embodiments, the 3′ annealing region comprises three 2′-fluoro-ribonucleotides. In certain embodiments, the at least one 2′-fluoro-ribonucleotide is 2′-fluoro-riboguanine (2′fG). In certain embodiments, any non-2′fG ribonucleotides in the 3′ annealing region of the TSO are riboguanine (rG) ribonucleotides.

In certain embodiments, the 3′ annealing region of the TSO, in a 5′ to 3′ direction, is selected from the group consisting of: rG-rG-2′fG; rG-2′fG-2′fG; 2′fG-2′fG-2′fG; 2′fG-2′fG-2′fG-2′fG; rN-2′fG-2′fG; rI-2′fG-2′fG; and 5′NI-2′fG-2′fG. In certain embodiments, the 3′ annealing region of the TSO, in a 5′ to 3′ direction, is: rG-2′fG-2′fG. In certain embodiments, the 3′ annealing region of the TSO, in a 5′ to 3′ direction, is: 2′fG-2′fG-2′fG.

In certain embodiments, the 5′ adapter region of the TSO further comprises one or more of: a barcode sequence, a unique molecule identifier (UMI), an amplification primer sequence, a sequencing primer sequence, a capture primer sequence, a sequence-specific nuclease cleavage site, a modified nucleotide, a biotinylated nucleotide, and a 5′ modification.

Aspects of the present disclosure include a kit comprising a template switching oligonucleotide (TSO) as described herein (e.g., a TSO comprising a 5′ adapter region and a 3′ annealing region, wherein the 3′ annealing region is configured to anneal to a 3′ overhang of a cDNA strand of an RNA-cDNA intermediate, wherein the TSO is capable of serving as a template for extension of the 3′ end of the cDNA strand, and wherein the 3′ annealing region comprises at least one 2′-fluoro-ribonucleotide). In certain embodiments, the kit further comprises a cDNA synthesis primer. In certain embodiments, the cDNA synthesis primer comprises a 5′ adapter region and a 3′ RNA annealing region. In certain embodiments, the 5′ adapter region of the cDNA synthesis primer comprises one or more of: a barcode sequence, a unique molecule identifier (UMI), an amplification primer sequence, a sequencing primer sequence, a capture primer sequence, a sequence-specific nuclease cleavage site, a modified nucleotide, a biotinylated nucleotide, and a 5′ modification. In certain embodiments, the 3′ RNA annealing region of the cDNA synthesis primer comprises a poly-T sequence. In certain embodiments, the 3′ RNA annealing region of the cDNA synthesis primer comprises a sequence complementary to at least one target RNA. In certain embodiments, the kit further comprises reagents for performing a cDNA synthesis reaction.

### BRIEF DESCRIPTION OF THE DRAWINGS

FIG. 1 schematically illustrates a general method for addition of a 3′ adapter to a cDNA of a poly-A mRNA template using a template-switching approach.

FIG. 2 depicts the general structures of a deoxyribonucleotide, ribonucleotide, LNA nucleotide analog, 2′-O-methyl ribonucleotide, and 2′-fluoro-ribonucleotide incorporated into a nucleic acid. Wavy lines indicate where each nucleotide is attached to a previous or subsequent base in the polynucleotide chain.

FIG. 3 schematically depicts an exemplary TSO having a 5′ adapter region and a 3′ annealing region that includes various combinations of ribonucleotides and 2′-fluoro-ribonucleotides.

FIG. 4 shows a graph comparing results for the total amount of cDNA generated using different TSOs in the top panel. The bottom panel shows the results of sequence analysis of the resulting cDNAs.

FIG. 5 illustrates the effects of varying the time of addition of a TSO to a cDNA synthesis reaction. The top panel shows a graph of total cDNA produced from experiments in which one of three different TSOs was added after 0, 30, 45, or 60 minutes. The bottom panel shows the percentage of full length non-chimeric (FLNC) reads obtained at various timepoints with the different TSOs.

FIG. 6 illustrates the effects of cleaning the cDNA prior to amplification. The top panel shows the cDNA yield from reactions performed with and without cDNA clean-up. The bottom panel shows the percentage of FLNC reads, as well as the percentages of 5′-5′ TSO and 3′-3′ RT primer reads (representing undesired products).

Schematic figures are not necessarily to scale.

### DETAILED DESCRIPTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, phage display, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual (all from Cold Spring Harbor Laboratory Press); Stryer, L. (1995) Biochemistry (4th Ed.) Freeman, N.Y., Gait; “Oligonucleotide Synthesis: A Practical Approach” 1984, IRL Press, London; Nelson and Cox (2000); Lehninger, Principles of Biochemistry 3rd Ed., W. H. Freeman Pub., New York, N.Y.; Berg et al. (2002) Biochemistry, 5th Ed., W. H. Freeman Pub., New York, N.Y.; Sambrook et al. Molecular Cloning—A Laboratory Manual (3rd Ed.), Vol. 1-3, Cold Spring Harbor Laboratory, Cold Spring Harbor, N.Y., 2000; and Ausubel et al., eds. Current Protocols in Molecular Biology, F.M., Current Protocols, John Wiley & Sons, Inc. (supplemented through 2020), all of which are herein incorporated in their entirety by reference for all purposes.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising), or alternatively including steps and compositions of no significance (consisting essentially of), or alternatively intending only the stated method steps or compositions (consisting of).

All numerical designations, e.g., pH, temperature, time, concentration, and molecular weight, including ranges, are approximations which, unless otherwise indicated, can vary ±0.1 unit. It is to be understood, although not always explicitly stated, that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

By “nucleic acid” or “polynucleotide” or grammatical equivalents herein is meant at least two nucleotides covalently linked together. A nucleic acid of the present invention will generally contain phosphodiester bonds, although in some cases, nucleic acid analogs are included that may have alternate backbones, comprising, for example, phosphoramide, phosphorothioate, phosphorodithioate, and peptide nucleic acid backbones and linkages. Other analog nucleic acids include those with positive backbones, non-ionic backbones, and non-ribose backbones, including those described in U.S. Pat. Nos. 5,235,033 and 5,034,506.

By “template switching oligonucleotide” or “TSO” (also referred to as a “template switch oligonucleotide”) is meant an oligonucleotide template to which a polymerase switches from an initial template (e.g., a template mRNA as described herein) during a nucleic acid polymerization reaction. A TSO may include one or more nucleotides (or analogs thereof) that are modified or otherwise non-naturally occurring. For example, the template switching oligonucleotide may include one or more nucleotide analogs (e.g., LNA, FANA, 2′-O-methyl ribonucleotides, 2′-fluoro ribonucleotides, or the like), linkage modifications (e.g., phosphorothioates, 3′-3′ and 5′-5′ reversed linkages), 5′ and/or 3′ end modifications (e.g., 5′ and/or 3′ amino, biotin, DIG, phosphate, thiol, dyes, quenchers, etc.), one or more fluorescently labeled nucleotides, or any other feature that provides a desired functionality to the template switching oligonucleotide.

As used herein, an “oligonucleotide” is a single-stranded multimer of nucleotides from 2 to 500 nucleotides in length, e.g., 2 to 200 nucleotides. Oligonucleotides may be synthetic or may be made enzymatically, and, in some embodiments, are 10 to 50 nucleotides in length. Oligonucleotides may contain ribonucleotide monomers or modified forms thereof, deoxyribonucleotide monomers or modified forms thereof, or a combination of ribo- and deoxyribo-nucleotide monomers or modified forms thereof, e.g., as certain TSOs described herein. Oligonucleotides may be 10 to 20, 21 to 30, 31 to 40, 41 to 50, 51-60, 61 to 70, 71 to 80, 80 to 100, 100 to 150 or 150 to 200, up to 500 or more nucleotides in length, for example.

As used herein, a “substantially identical” nucleic acid is one that has at least 80%, 85%, 90%, 95%, 96%, 97%, 98% or 99% sequence identity to a reference nucleic acid sequence. The length of comparison is preferably the full length of the nucleic acid, but is generally at least 20 nucleotides, 30 nucleotides, 40 nucleotides, 50 nucleotides, 75 nucleotides, 100 nucleotides, 125 nucleotides, or more.

As used herein, the term “reverse transcriptase” is defined as an enzyme that catalyzes the formation of DNA from an RNA template. In many aspects of the present disclosure, a reverse transcriptase is a DNA polymerase that can be used for first-strand cDNA synthesis from an RNA template. Any RNA template may be used, including messenger RNA (mRNA), microRNA (miRNA), small interfering RNA (siRNA), Piwi-interacting RNA (piRNA), small nuclear RNA (snRNA), small non-coding RNA (sncRNA), long non-coding RNA (lncRNA), circulating free RNA (cfRNA), circulating tumor RNA (ctRNA), ribosomal RNA (rRNA), viral RNA, total RNA, etc.

As used herein, two sequences (or subregions of two larger sequences) are said to be “fully complementary” or “perfectly complementary” to one another if they are capable of hybridizing to one another to form antiparallel, double-stranded nucleic acid structure in which each base of a first strand of the double-stranded nucleic acid structure forms hydrogen bonds with its corresponding base of the second strand. For example, for naturally occurring DNA, dA is complementary to dT and dC is complementary to dG. Thus, the DNA sequence 5′-AGCT-3′ is fully complementary to the DNA sequence 5′-AGCT-3′. It is noted that two sequences need not be fully complementary to hybridize to one another. The conditions necessary for hybridization of two nucleic acid strands (the temperature, incubation time, and buffer components, generally referred to as the “stringency” of a hybridization reaction) is routinely determined by those of skill in the art and is based in part on the length of the hybridization region, the level of complementarity between the two strands within the hybridization region, and the complexity of the sample.

In describing aspects of the methods disclosed herein, reference will be made to the Figures. It is to be understood that the Figures merely illustrate specific embodiments of the disclosed methods and are not intended to be limiting.

### General Template Switch Method

The subject disclosure is drawn to compositions and methods for improving the generation of cDNAs from an RNA template using a Template Switching Oligonucleotide (TSO). The resulting cDNAs include a 3′ adapter sequence. In certain embodiments, the compositions and methods detailed herein are useful in generating full-length cDNA copies of poly-A mRNA templates from samples containing low quantities of quantities of total or poly A+ RNA, including from samples containing from 1 picogram (pg) to 5 micrograms (μg) of total RNA. In some embodiments, RNA from a single cell is used as the RNA template. mRNA can typically be isolated from almost any source using protocols and methods described in the literature, e.g., Sambrook and Ausubel, as well as commercially available mRNA isolation kits, e.g., the RNeasy Mini Kit (Qiagen), the mRNA-ONLY™ Prokaryotic mRNA Isolation Kit and the mRNA-ONLY™ Eukaryotic mRNA Isolation Kit (Epicentre Biotechnologies), the FastTrack 2.0 mRNA Isolation Kit (Invitrogen), and the Easy-mRNA Kit (BioChain). In addition, mRNA from various sources, e.g., bovine, mouse, and human, and tissues, e.g. brain, blood, and heart, is commercially available from, e.g., BioChain (Hayward, Calif.), Ambion (Austin, Tex.), and Clontech (Mountainview, Calif.).

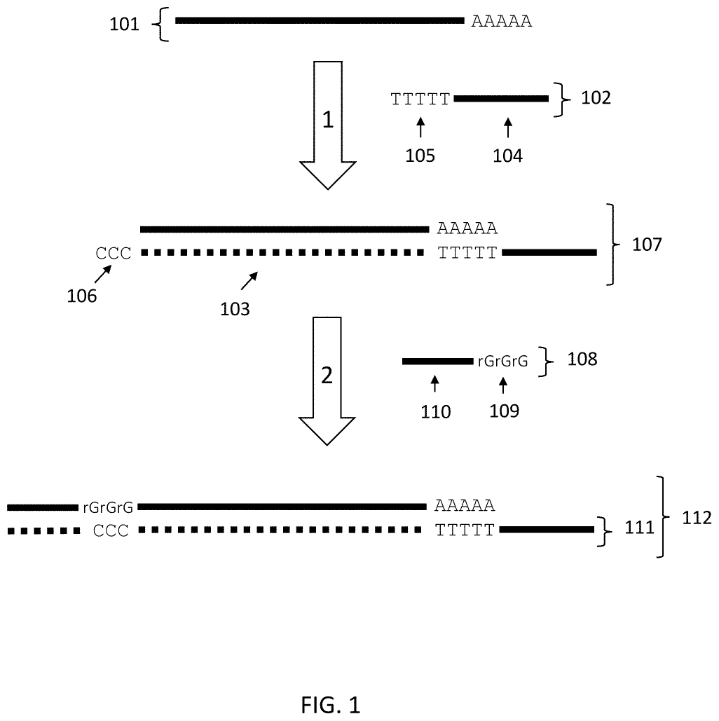

FIG. 1 shows a schematic illustration of a general method for addition of a 3′ adapter to cDNA of a poly-A mRNA template, e.g., for cDNA library preparation, using a template-switching approach. General methods for preparing such cDNAs can be found in, e.g., U.S. Pat. No. 5,962,272, entitled “Method and compositions for full-length cDNA cloning using a template-switching oligonucleotide”; U.S. Pat. No. 9,410,173, entitled “Template switch-based methods for producing a product nucleic acid”; and US Patent Application Publication No. 2018/0037884, entitled “Methods and compositions for preventing concatemerization during template-switching”; each of which are hereby incorporated herein by reference in their entirety.

In Step 1 in FIG. 1, a sample containing poly-A mRNA 101 is provided and combined with a cDNA synthesis primer 102 under conditions that allows hybridization of the cDNA synthesis primer to a cognate site in the mRNA template and synthesis of a cDNA strand 103 from the hybridized cDNA synthesis primer. In general, the mRNA/cDNA synthesis primer mixture will include a reverse transcriptase, dNTPs (a combination of dATP, dCTP, dTTP, and dGTP), and buffer components that promote reverse transcription. The cDNA synthesis primer in FIG. 1 includes two domains: (i) 5′ adapter region 104, and (ii) 3′ mRNA hybridization region 105 (sometimes referred to as a priming region). It is noted here that in certain embodiments the cDNA synthesis primer does not include domain 104, the 5′ adapter region, and can thus consist of or comprise only a priming region. As such, the 5′ adapter region 104 is an optional element that is employed at the discretion of the user. Further, while the mRNA hybridization region 105 in FIG. 1 is shown as a poly-T sequence that is designed to hybridize to the poly-A tail of the mRNAs in the sample, other sequences designed to hybridize to other known regions in one or more RNA templates in the sample could be used. The reverse transcriptase synthesizes a first strand cDNA to the 5′ end of the mRNA template and, in this example, adds three non-templated dC residues to the 3′ end of the first strand cDNA thereby creating a 3′ overhang region 106. (It is noted that in some embodiments, the polymerase may be capable of incorporating any number of non-templated bases, including 1, 2, 3, 4, 5, 6, 7, 8, 9, 10 or more additional nucleotides at the 3′ end of the nascent cDNA strand). This process results in a first mRNA/cDNA complex 107. A variety of DNA polymerases possessing reverse transcriptase activity and terminal transferase activity can be used in this step. Examples include the DNA polymerases derived from organisms such as thermophilic bacteria and archaebacteria, retroviruses, yeast, Neurospora, Drosophila, primates and rodents. In some embodiments, the DNA polymerase is isolated from Moloney murine leukemia virus (M-MLV) (e.g., as described in U.S. Pat. No. 4,943,531, hereby incorporated herein by reference in its entirety) or M-MLV reverse transcriptase lacking RNaseH activity (e.g., as described in U.S. Pat. No. 5,405,776, hereby incorporated herein by reference in its entirety), human T-cell leukemia virus type I (HTLV-I), bovine leukemia virus (BLV), Rous sarcoma virus (RSV), human immunodeficiency virus (HIV), Thermus aquaticus (Taq), or Thermus thermophilus (Tth) (e.g., as described in U.S. Pat. No. 5,322,770, hereby incorporated herein by reference in its entirety). These DNA polymerases may be isolated from an organism itself or, in some cases, obtained commercially. DNA polymerases useful with the subject invention can also be obtained from cells expressing cloned genes encoding the polymerase. Suitable reaction conditions for use of various reverse transcriptases are well known in the art.

It is noted here that while a single DNA polymerase may be used to generate cDNA strand 103 and overhang region 106, in certain embodiments of the preset disclosure synthesis of cDNA strand 103 is performed by a DNA polymerase and the addition of the overhang region 106 is performed by a separate enzyme having 3′ terminal transferase activity. Examples of such enzymes include, but are not limited to: terminal deoxynucleotidyl transferase (TdT), DNA polymerase θ, Klenow Fragment (3′→5′ exo-), Taq DNA polymerase, and the like.

In Step 2 in FIG. 1, a template switching oligonucleotide (TSO) 108 is combined with the mRNA/cDNA complex that includes (i) 3′-terminal nucleotide sequence (also called an annealing region), here exemplified by three riboguanine residues (rGrGrG) 109, which can anneal to the 3′ overhang 106 of the cDNA strand of the mRNA/cDNA complex 107 and (ii) 5′ adapter region 110. Once the TSO 108 is annealed to 3′ overhang region 106, the reverse transcriptase “switches” from copying the mRNA template 101 to copying the TSO 108, with the non-templated nucleotides of the 3′ overhang 106 in between. This results in a single contiguous cDNA strand 111 that contains, in a 5′ to 3′ orientation: (i) adapter region 104, (ii) the poly-T region, (iii) a complement of the mRNA template, (iv) the non-templated nucleotides, and (v) a complement of adapter region 110 (which complement can be referred to as a 3′ adapter region on the cDNA). This cDNA strand 111 can remain hybridized to the template mRNA in a second mRNA/cDNA complex 112.

As noted above, while the starting RNA in the embodiment shown in FIG. 1 is an mRNA, any RNA of interest may be used as a starting template. In some of these embodiments, the 3′ poly-A region is not present and thus the cDNA synthesis primer employed will need to be designed to prime at a different desired location (e.g., the sequence at the 3′ end of the RNA templates or other desired internal sequence of the RNA templates). In further embodiments, nucleotides can be added to the 3′ end of the RNA templates to generate the region to which the cDNA synthesis primer anneals. Any convenient method for adding a region of known sequence to the 3′ end of a template RNA may be employed, e.g., the addition of desired polynucleotides using one or more template-independent polymerases (see, Georges Martin and Walter Keller 2007 “RNA-specific ribonucleotide transferases” RNA 13:1834-1849, the entirety of which is hereby incorporated herein by reference in its entirety) or by ligation of a synthetic oligonucleotide. No limitation on this regard is intended.

While the embodiment in FIG. 1 is shown for a single RNA template, the process is most generally used on samples containing a population of RNA templates, e.g., a population of RNA templates derived from a source or sources of interest (as discussed elsewhere herein).

Once obtained, cDNA strand 111 can be used in any downstream process of interest to a user.

For example, in certain embodiments, cDNA strand 111 is subjected to an amplification reaction, e.g., using one or more amplification primers specific for sequences in the adapter regions 104 and 110. Such amplification processes, e.g., PCR (polymerase chain reaction), isothermal amplification, and the like, can generate products useful for downstream applications, e.g., sequencing (discussed in further detail elsewhere herein).

### Modified Template Switching Oligonucleotides (TSOs) and Methods of Use

The present disclosure provides modified TSOs and methods for using the same for generating 3′ adapter-containing cDNAs from RNA templates. Specifically, modified TSOs of the present disclosure include at least one 2′-fluoro-ribonucleotide in the 3′ annealing region. In certain embodiments, methods for using such modified TSOs include adding them to the cDNA synthesis reaction after an incubation period, e.g., from about 10 minutes to 4 hours or more. As described herein, the inclusion of one or more 2′-fluoro-ribonucleotides in the 3′ annealing region of the modified TSO results in an increase in the amount of 3′ adapter-containing cDNA product produced in the reaction. This is particularly useful with samples having low input levels of RNA, e.g., from about 1 pg to 1 μg or RNA from a single cell.

In view of the above, aspects of the present disclosure include methods for generating a complementary DNA (cDNA) strand with a 3′ adapter region by combining an RNA template with a cDNA synthesis primer, a TSO of the present disclosure, and a reverse transcriptase under cDNA synthesis conditions. The TSO includes a 5′ adapter region and a 3′ annealing region comprising at least one 2′-fluoro-ribonucleotide. The cDNA synthesis primer in the reaction is designed to anneal to the RNA template (via a 3′ priming region in the cDNA synthesis primer), and the reverse transcriptase extends the annealed cDNA synthesis primer, thereby producing an RNA-cDNA intermediate. The reverse transcriptase used in the reaction can be one that adds non-templated nucleotides to the 3′ end of the newly synthesized cDNA, and thus the cDNA strand of the RNA-cDNA intermediate has a 3′ overhang. However, in some embodiments, a second enzyme is included in the reaction mixture that serves the function of adding non-templated bases to the 3′ end of the cDNA strand, e.g., TdT, DNA polymerase θ, Klenow Fragment (3′→5′ exo-), Taq DNA polymerase, and the like. The 3′ overhang of the cDNA strand can have different compositions of bases, e.g., any combinations of dA, dG, dT and dC bases, and can be of varying length. However, in general, the 3′ overhang on the cDNA strand is from 2 to 6 bases in length, e.g., 2, 3, 4, 5, or 6 bases, and is primarily comprised of dC residues. While for many embodiments the 3′ overhang will be considered to have 3 dC bases (dC-dC-dC), modified TSOs that take into consideration variations in the 3′ overhang configuration in the cDNA are contemplated. After production of the cDNA strand with its 3′ overhang, the 3′ annealing region of the modified TSO (i.e., containing at least one 2′-fluoro-ribonucleotide) anneals to the 3′ overhang of the RNA-cDNA intermediate, and the reverse transcriptase extends the 3′ end of the cDNA strand of the RNA-cDNA intermediate using the annealed TSO as a template. While not being bound by theory, it appears that the presence of the at least one 2′-fluoro-ribonucleotide in the annealing region of the TSO improves annealing of the TSO to the 3′ overhang of the cDNA strand, thereby improving cDNA yield.

### Examples of Modified TSOs

As noted above, TSOs for use in generating cDNAs with 3′ adapter regions include at least two regions: (i) a 5′ adapter region and (ii) a 3′ annealing region (elements 110 and 109, respectively, as described in FIG. 1). The 3′ annealing region of the modified TSOs of the present disclosure includes at least one 2′-fluoro-ribonucleotide in the 3′ annealing region.

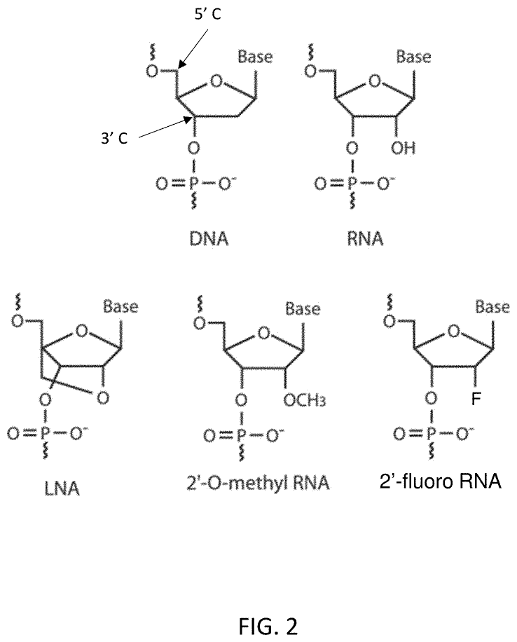

FIG. 2 shows structures of several nucleotide species that are discussed in this disclosure: a deoxyribonucleotide, a ribonucleotide, a locked-nucleic acid (LNA) nucleotide analog, a 2′-O-methyl ribonucleotide, and a 2′-fluoro-ribonucleotide. The wavy lines indicate where each nucleotide is attached to a previous base (through the oxygen attached to the 5′ C) or a subsequent base (through the phosphate attached to the 3′ C) when present in a polynucleotide chain.

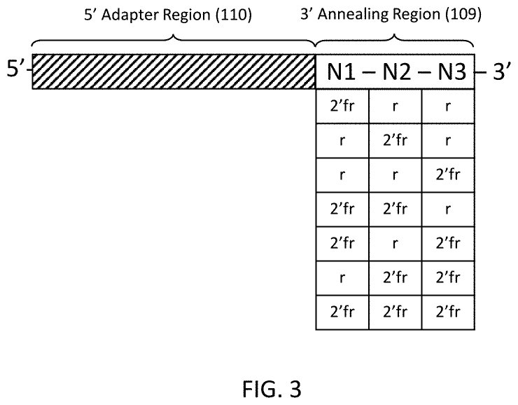

FIG. 3 shows an exemplary TSO structure having a 5′ adapter region 110 and a 3′ annealing region 109. While the annealing region of a TSO can include, e.g., from 3 to 6 nucleotide residues, as noted above, the TSO in FIG. 3 has a 3′ annealing region comprising three ribonucleotide residues designated N1-N2-N3 in a 5′ to 3′ orientation. At least one of N1 to N3 is a 2′-fluoro-ribonucleotide. Therefore, the annealing region 109 in FIG. 3 can have one 2′-fluoro-ribonucleotide, two 2′-fluoro-ribonucleotides, or three 2′-fluoro-ribonucleotides. FIG. 3 shows the possible orientations of the ribonucleotide(s) (r in FIG. 3) and 2′-fluoro-ribonucleotide(s) (2′fr in FIG. 3) in the annealing region with respect to N1 to N3. As shown, the 3 nucleotide annealing region shown in FIG. 3 can have one 2′-fluoro-ribonucleotide (N1, N2, or N3 is a 2′-fluoro-ribonucleotide), two 2′-fluoro-ribonucleotides (N1 and N2, N1 and N3, or N2 and N3 are 2′-fluoro-ribonucleotides), or three 2′-fluoro-ribonucleotides (N1, N2, and N3 are 2′-fluoro-ribonucleotides).

In certain embodiments, the 2′-fluoro-ribonucleotides in the annealing region are 2′-fluoro-riboguanine (2′fG) residues (also referred to as 2′-fluoro-riboguanosines). In some of these embodiments, any non-2′fG ribonucleotides in the 3′ annealing region of the TSO are riboguanine (rG) ribonucleotides (also referred to as riboguanosines).

While the use of 2′fG and rG residues in the annealing region are preferred in some embodiments, other 2′-fluoro-ribonucleotides and ribonucleotides may be used. For example, the 3′ annealing region can include riboadenine (rA), ribocytosine (rC), and/or ribouracil (rU), or their 2′-fluoro-modified counterparts. In some embodiments, the 3′ annealing region comprises a universal nucleotide base, e.g., riboinosine (rI) and/or 5′ 5-nitroindole (5′NI). In still further embodiments, the TSO can be synthesized such that the 3′ annealing region includes a degenerate ribonucleotide base (rN), resulting in a TSO population in which the rN is one of a selection of desired ribonucleotides (e.g., rA, rC, rG, and rU, or any desired combination thereof).

In certain embodiments, the 3′ annealing region of the TSO, in a 5′ to 3′ direction, is one of the following: rG-rG-2′fG; rG-2′fG-2′fG; 2′fG-2′fG-2′fG; 2′fG-2′fG-2′fG-2′fG; rN-2′fG-2′fG; rI-2′fG-2′fG; and 5′NI-2′fG-2′fG.

In addition to the 3′ annealing region, TSOs include a 5′ adapter region. This region can include any sequence that a user desires to be attached to the 3′ end of a cDNA using the methods described herein. Similarly, the cDNA synthesis primer employed in the cDNA synthesis reaction can, in certain embodiments, include any desired sequence/modification that a user desires to be attached to the 5′ end of a cDNA. No limitation for the sequence of this domain, or modifications present in it, is intended.

In certain embodiments, the 5′ adapter region of the TSO comprises one or more of: a barcode sequence, a unique molecule identifier (UMI), an amplification primer sequence, a sequencing primer sequence, a nanopore sequencing adapter, a capture primer sequence (or other capture moiety), a sequence-specific nuclease cleavage site, a modified nucleotide, a biotinylated nucleotide, and a 5′ modification. In some embodiments, the 5′ adaptor region comprises at least a barcode sequence and an amplification primer sequence. The 5′ adaptor region optionally comprises at least two nucleotides, e.g., 2-100 nucleotides or 5-50 nucleotides.

In general, a barcode sequence is a nucleotide sequence that is used to positively identify a sample from which a particular nucleic acid or copy thereof was derived (in this case an RNA template/cDNA). For example, if 5 different RNA samples were subjected to 5 different cDNA synthesis reactions as described herein, the TSO used for each of the 5 different reactions can include a barcode sequence in the 5′ adapter region that is different from all of the barcode sequences in the other 4 TSOs. Exemplary useful barcodes are known in the art (see, e.g., 384 barcode sequences available at github (dot) com/PacificBiosciences/Bioinformatics-Training/wiki/Barcoding), and additional barcodes can be designed if desired.

In general, a unique molecule identifier (UMI) is a sequence of nucleotides used to distinguish individual nucleic acid molecules from one another. UMIs may be sequenced (or otherwise detected) along with the nucleic acid molecules with which they are associated to determine whether the read sequences are those of one source DNA molecule or another.

In general, an amplification primer sequence is a nucleic acid sequence designed to provide a site to which a nucleic acid synthesis primer anneals to initiate nucleic acid synthesis by a polymerase, e.g., for linear or non-linear amplification of a nucleic acid, e.g., in PCR. An amplification primer sequence or the complement thereof and its cognate amplification primer thus have sufficient complementarity to hybridize under the conditions of the amplification reaction performed. For the example shown in FIG. 1, PCR can be performed, e.g., using a forward primer comprising sequence from adapter region 110 (complementary to cDNA strand 111, which includes a complement of adapter region 110) and a reverse primer comprising sequence from adapter region 104.

In general, a sequencing primer sequence is a nucleic acid sequence designed to provide a site to which a sequencing primer anneals to initiate a sequencing by synthesis (SBS) reaction. A sequencing primer sequence or the complement thereof and its cognate sequencing primer thus have sufficient complementarity to hybridize under the conditions of the sequencing reaction performed.

In general, a nanopore sequencing adapter is a nucleic acid sequence used in a nanopore sequencing process and may include additional bound components that facilitate nanopore sequencing reactions, e.g., bound enzymes (e.g., helicases, polymerases, or other motor proteins), membrane binding moieties (e.g., cholesterol), and the like.

In general, a capture primer sequence is a nucleic acid sequence designed to provide a site to which a capture primer anneals for the purpose of isolating adapter-associated nucleic acids from non-adapter associated nucleic acids, e.g., by immobilizing the capture primer to a solid surface or substrate. A capture primer sequence or the complement thereof and its cognate capture primer thus have sufficient complementarity to hybridize under the conditions of the isolation process performed. It is noted that non-nucleic acid based capture moieties can be attached to a TSO (or other oligonucleotides, e.g., a cDNA synthesis primer) for use in isolating nucleic acids attached thereto where in some embodiments the capture moiety is a member of a binding pair (e.g., biotin, avidin, streptavidin, digoxigenin, antibody binding domain, antigen, etc.). For example, the capture moiety can be in the adapter region in the form of a biotinylated nucleotide; the resulting adapter-containing cDNA can then be isolated by binding to avidin or streptavidin.

In general, a sequence-specific nuclease cleavage site is a nucleic acid sequence that is a recognition site for a cognate nuclease that recognizes that sequence and cleaves the nucleic acid (either one or both strands), e.g., a restriction enzyme, nickase, a uracil-specific excision reagent (USER) that generates a single nucleotide gap at the site of a uracil base, or an engineered nuclease/nickase. Examples of engineered nucleases/nickases include, but are not limited to, RNA-directed endonucleases (e.g., the CRISPR-Cas system, e.g., Cas9 and Cpf1 DNA endonucleases), artificial restriction enzymes (e.g., TAL Effector Nucleases (TALENs), zinc-finger nucleases (ZFNs)), and variants thereof.

In general, a modified nucleotide is one that has a modified chemical structure as compared to DNA or RNA nucleotides (e.g., methylated bases, PNA (peptide nucleic acid) nucleotides, LNA (locked nucleic acid) nucleotides, 2′-O-methyl-modified nucleotides, 2′-fluoro-modified nucleotides, and the like).

In general, a 5′ modification is any modification to the 5′ terminus of an adapter region of a TSO. For example, the 5′ terminus can be modified to protect it from nuclease degradation or to allow its detection, e.g., by attachment to a detectable moiety, e.g., a fluorescent dye.

It is noted here that similar to the TSO of the present disclosure, in embodiments in which the cDNA synthesis primer includes its own 5′ adapter region (in addition to its 3′ RNA annealing region), this second 5′ adapter region can also include one or more of: a barcode sequence, a unique molecule identifier (UMI), an amplification primer sequence, a sequencing primer sequence, a nanopore sequencing adapter, a capture primer sequence (or other capture moiety), a sequence-specific nuclease cleavage site, a modified nucleotide, a biotinylated nucleotide, and a 5′ modification (similar to the 5′ adapter region of the TSO). Any such barcode sequence, primer sequence, and/or other feature of the cDNA synthesis primer can independently be the same as or different than those on the TSO.

In some embodiments, the 5′ adapter region of the TSO includes a first amplification primer sequence and the 5′ adapter region of the cDNA synthesis primer (the second 5′ adapter region) includes a second amplification primer sequence. In such embodiments, cDNA produced in the TSO reaction, which includes a complement of the 5′ adapter region of the TSO at its 3′ end, can be amplified by performing a PCR with a primer pair specific for the first and second amplification primer sequences. While the first and second amplification primer sequences are located at opposite ends of the product of the cDNA synthesis reaction, they can be designed to allow amplification using a single amplification primer, e.g., by performing a PCR. In other embodiments, the first and second amplification primer sequences are designed to anneal to amplification primers having different sequences, and as such two different amplification primers are needed to amplify the product by PCR. Adjusting such sequences are up to the desires of the user.

In some embodiments, the TSO includes a modification that prevents a polymerase from switching from the TSO to a different template nucleic acid after synthesizing the complement of the 5′ end of the TSO (e.g., a 5′ adapter sequence of the TSO). Useful modifications include, but are not limited to, an abasic lesion (e.g., a tetrahydrofuran derivative), a nucleotide adduct, an iso-nucleotide base (e.g., isocytosine, isoguanine, and/or the like), and any combination thereof.

In additional embodiments, the TSO includes a 3′ modification or modified nucleotide that renders it incapable of being used as a nucleic acid synthesis primer (i.e., it cannot initiate nucleic acid synthesis when annealed to a template polynucleotide). For example, the TSO can include a 3′-deoxy nucleotide species (e.g., a di-deoxy nucleotide, a 2′-fluoro-3′-deoxy nucleotide, e.g., a 2′-fluoro-3′-deoxyriboguanosine, and the like).

Oligonucleotides, including oligonucleotides for use as TSOs or primers, can be synthesized using techniques well known in the art or can be purchased from any of a variety of commercial suppliers.

### RNA Templates

Any RNA template of interest may be employed to generate a cDNA product according to the methods described herein. Examples of RNA templates include, but not are limited to: messenger RNA (mRNA), non-coding RNA, microRNA (miRNA), small interfering RNA (siRNA), Piwi-interacting RNA (piRNA), small nuclear RNA (snRNA), small non-coding RNA (sncRNA), long non-coding RNA (lncRNA), circulating free RNA (cfRNA), circulating tumor RNA (ctRNA), ribosomal RNA (rRNA), viral RNA, and total RNA.

In certain embodiments, the RNA templates are enriched prior to performing the methods of the present disclosure. Any of the RNA templates listed above can be enriched for prior to use in the methods disclosed herein. Moreover, any convenient enrichment process for these different RNA template can be used, including positive or negative selection methods. Enrichment can be based on any desired feature of the RNA templates, including the size/length of the templates (generally referred to as size-selection) and/or the presence or absence of a particular nucleotide sequence, domain, or modification in the templates. For example, a capture moiety specific for a sequence, domain, or modification can be employed to enrich for, or deplete, particular RNA templates from a parent sample. Capture moieties include sequence specific RNA binding proteins, oligonucleotide primers complementary to specific RNA sequences, aptamers, antibodies, etc. No limitation in this regard is intended. In some embodiments, undesired RNA templates in an RNA sample can be digested or degraded using one or more nucleases.

In many embodiments, the RNA template is an mRNA template. The mRNA can be in an RNA sample obtained directly from a source and thus be present with significant amounts of other types of RNA (e.g., rRNA). Alternatively, the mRNA can be from sample that has been enriched for mRNAs, e.g., by selecting for RNAs with 3′ poly-A tails or having a particular size or coding sequence. In many embodiments, the mRNA has a poly-A tail at the 3′-end. In additional embodiments, the mRNA has a 7-methylguanosine CAP structure at the 5′-end.

It is also contemplated that the cDNAs generated according to the methods described herein (i.e., using modified TSOs as described) can be enriched prior to any downstream analysis (e.g., sequence analysis). Any convenient enrichment method may be employed, including methods similar to those outlined above for enriching for RNA templates of interest (e.g., the use of capture moieties, nucleases, size-selection, etc.).

### Methods of Using Modified TSOs

The present disclosure describes improved methods for generating a cDNA strand using a TSO having at least one 2′-fluoro-ribonucleotide in its 3′ annealing region, as described in detail above.

In some embodiments, the method includes combining an RNA template with a cDNA synthesis primer (as described above) and a reverse transcriptase under cDNA synthesis conditions such that the cDNA synthesis primer anneals to the RNA template and the reverse transcriptase generates an RNA-cDNA intermediate from the annealed cDNA synthesis primer. The cDNA strand of the RNA-cDNA intermediate includes a 3′ overhang added by the reverse transcriptase (or optionally by a second enzyme in the reaction mixture, also as described above). A TSO (preferably a modified TSO as described herein) combined with the reaction, either simultaneously with the cDNA synthesis primer or after a pre-extension period, anneals to the 3′ overhang of the RNA-cDNA intermediate, via a 3′ annealing region on the TSO, and the reverse transcriptase extends the 3′ end of the cDNA strand of the RNA-cDNA intermediate using the annealed TSO as a template. A complement of the 5′ adapter region on the TSO is thus added to the 3′ end of the cDNA strand to generate a cDNA with a 3′ adapter region.

As indicated, in certain embodiments the cDNA synthesis primer and the TSO are simultaneously combined with the RNA template and reverse transcriptase. In alternate embodiments, the cDNA synthesis primer and the reverse transcriptase are combined with the RNA template under cDNA synthesis conditions to form a pre-extension mixture to generate the RNA-cDNA intermediate prior to combining with the TSO. The pre-extension mixture can be incubated for any amount of time desired to allow for completion of cDNA synthesis (production of the RNA-cDNA intermediate) prior to combining with the TSO, e.g., from 5 minutes to 24 hours, including from 10 minutes to 4 hours, from 30 minutes to 2 hours, or for about 1 hour.

A particular embodiment of the present disclosure includes obtaining a sample comprising mRNAs having 3′ poly-A tails, producing a cDNA synthesis reaction by contacting the sample with a cDNA synthesis primer having a 3′ poly-T annealing region and a reverse transcriptase under cDNA synthesis conditions, allowing the cDNA synthesis reaction to proceed for from 5 minutes to 24 hours to produce cDNAs with 3′ overhangs, adding a TSO having a 5′ adapter region and a three-nucleotide 3′ annealing region with at least one 2′-fluoro-riboguanine (2′fG) nucleotide, and incubating the cDNA synthesis reaction under conditions that allow the 3′ annealing region of the TSO to anneal to the 3′ overhangs of the cDNAs and extend the 3′ end of the cDNAs using the annealed TSO as a template, thereby generating adapter-containing cDNAs.

In any of the methods described herein, the 3′ annealing region of the TSO can include one 2′fG nucleotide, two 2′fG nucleotides, or three 2′fG nucleotides. In addition, in some embodiments, any non-2′fG nucleotides in the 3′ annealing region of the TSO are riboguanine (rG) nucleotides. While the use of 2′fG and rG residues in the annealing region are preferred in some embodiments, other 2′-fluoro-ribonucleotides and ribonucleotides may be used. For example, the 3′ annealing region can include riboadenine (rA), ribocytosine (rC), and/or ribouracil (rU), or their 2′-fluoro-modified counterparts. In some embodiments, the 3′ annealing region comprises a universal nucleotide base, e.g., riboinosine (rI) and/or 5′ 5-nitroindole (5′NI). In still further embodiments, the TSO can be synthesized such that the 3′ annealing region includes a degenerate ribonucleotide base (rN), resulting in a TSO population in which the rN is one of a selection of desired ribonucleotides (e.g., rA, rC, rG, and rU, or any desired combination thereof). Examples of 3′ annealing regions of the TSOs described herein include, in a 5′ to 3′ direction: rG-rG-2′fG; rG-2′fG-2′fG; 2′fG-2′fG-2′fG; 2′fG-2′fG-2′fG-2′fG; rN-2′fG-2′fG; rI-2′fG-2′fG; and 5′NI-2′fG-2′fG.

As described above, the 5′ adapter region of the TSO further can include sequences that find use to a user in a downstream application. Non-limiting examples include one or more of: a barcode sequence, a unique molecule identifier (UMI), an amplification primer sequence, a sequencing primer sequence, a nanopore sequencing adapter, a capture primer sequence (or other capture moiety), a sequence-specific nuclease cleavage site, a modified nucleotide, a biotinylated nucleotide, and a 5′ modification. The cDNA synthesis primer can also include a 5′ adapter region (a “second” 5′ adapter region, with the 5′ adapter region of the TSO being the “first” 5′ adapter region) in addition to its 3′ RNA annealing region. As with the first 5′ adapter region, the second 5′ adapter region can include one or more of: a barcode sequence, a unique molecule identifier (UMI), an amplification primer sequence, a sequencing primer sequence, a nanopore sequencing adapter, a capture primer sequence (or other capture moiety), a sequence-specific nuclease cleavage site, a modified nucleotide, a biotinylated nucleotide, and a 5′ modification. Any such barcode sequence, primer sequence, and/or other feature of the second 5′ adapter region can independently be the same as or different than those in the first 5′ adapter region.

In certain embodiments of the present disclosure, the template switch reaction is performed on individual cells or small cell populations, e.g., in assays designed to analyze gene expression at the single cell or small cell population level. These cells may be partitioned with the reaction components to perform a template switch reaction in any convenient manner, e.g., segregated or otherwise sorted (e.g., by flow cytometry) into wells of a microtiter plate or separate tubes, segregated into microfluidic droplets in an emulsion, etc. Such approaches are described in US Patent Publication No. US20150376609 assigned to 10× Genomics, Inc., and titled “Methods of Analyzing Nucleic Acids from Individual Cells or Cell Populations”, and US Patent Publication No. US20180030515 titled “Droplet-Based Method and Apparatus for Composite Single-Cell Nucleic Acid Analysis”, both of which are hereby incorporated herein by reference in their entirety. The use of barcodes and/or UMIs to aid in the analysis of gene expression from single cells (or different small populations of cells) is also discussed in these publications. Thus, the use of cDNA synthesis primers and/or TSOs with barcode and/or UMI sequences as described herein can be employed in these embodiments.

For example, mRNA from a single cell can be analyzed by co-partitioning the cell with (i) a bead having barcoded cDNA synthesis primers attached thereto, (ii) a modified TSO of the present disclosure, (iii) reverse transcriptase, and (iv) other reagents, e.g., for performing DNA synthesis, into a partition (e.g., a droplet in an emulsion). Once partitioned, the cell is lysed while the barcoded cDNA synthesis primers are released from the bead (e.g., via the action of a reducing agent included in the partition). The partitioned mixture is then placed under conditions in which the poly-T segment of the cDNA synthesis primer hybridizes to the poly-A tail of mRNA released from the cell and the template switch reaction can proceed, as detailed herein, resulting in the production of adapter-containing synthesis products (schematically shown in FIG. 1). All of the cDNA transcripts of the individual mRNA molecules in this partition will include a common barcode sequence, i.e., the barcode in the cDNA synthesis primer. Multiplexing this partitioning/template switch reaction using a 1:1 correspondence of a population of beads, each having a cDNA synthesis primer having a unique barcode, and a population of cells (i.e., so that a single cell is partitioned with a single bead) allows the analysis of mRNA expression on a cell-by-cell basis. In addition, a user could include a UMI in either the cDNA synthesis primer or the modified TSO such that cDNAs made from different mRNA molecules within a given partition will vary at this unique sequence. Further, in some embodiments, the modified TSO is not included in the emulsion droplets but rather added after the emulsion is broken (after cDNA synthesis) such that the cDNAs made from different cells have the same 3′ adapter region.

In addition to the barcode and/or UMI sequences in the cDNA synthesis primer and/or the modified TSO in the partitioned reactions noted above, other functional sequences can be present, including sequences for performing NGS sequencing reactions, e.g., Illumina sequencing reactions, or sequences that aid in generating templates for particular sequencing platforms, e.g., sites that facilitate ligation of sequencing adapters, e.g., hairpin adapters for SMRT™ Sequencing (single molecule real time sequencing) or adapters for nanopore sequencing.

### Downstream Processes and Analyses

The cDNAs produced according to the present disclosure find use in any number of downstream processes and/or analyses as desired by a user. As such, no limitation in this regard is intended. The examples below are provided merely for illustrative purposes.

In some embodiments, a method of the present disclosure includes amplifying the cDNA strand comprising the 3′ adapter region, e.g., linearly or exponentially (e.g., using PCR). Thus, in some embodiments, a second-strand cDNA synthesis reaction is performed to produce a single double-stranded DNA that includes the 5′ adapter regions of the TSO and cDNA synthesis primer, one on each end. This process may include degrading the original RNA template (e.g., using an enzyme with RNaseH activity). In some embodiments, the 5′ adapter regions of the TSO and/or cDNA synthesis primers can include an amplification primer sequence that serves as a binding site for one or more amplification primers. In cases in which the method includes performing a PCR on the cDNA generated, a primer pair specific for these amplification primer sequences (also called the “first” and “second” amplification primer sequences) can be used. The first and second amplification primer sequences may be different, requiring two different amplification primers for PCR, or the same, requiring only a single amplification primer for PCR. No limitation in this regard is intended.

In some embodiments, the cDNA product (either before or after amplification or second strand synthesis) can be ligated into a conventional vector (including a plasmid, cosmid, phage, or retroviral vector and so on) or to adapter(s) that find use in downstream analyses or processes, e.g., sequencing reactions, transformation into host cells, in vitro replication, and the like. In some embodiments, hairpin adapters, e.g., as used in generating SMRTbell™ templates (circular nucleic acids with a double-stranded central region and two hairpin ends; Pacific Biosciences of California, Inc.), are ligated to the ends of the cDNA product, e.g., after second strand synthesis/amplification. Production of such circular nucleic acids by ligation of stem-loop adapters is described, e.g., in U.S. Pat. No. 8,153,375 “Compositions and Methods for Nucleic Acid Sequencing” and in Travers et al. (2010) Nucl. Acids Res. 38(15):e159, each of which is incorporated herein by reference in its entirety for all purposes. Where adapters are ligated to the cDNA product (or amplicons thereof), the ends can be treated to be compatible with ligation, e.g., rendered blunt-ended, digested with a restriction enzyme(s) that leave ends that are compatible with the ends of the adapters, etc. as is well known in the art. No limitation in this regard is intended.

Prior to any downstream process or analysis, the cDNA product generated according to the methods described herein can be subjected to a purification step. This step can remove excess primers, nucleotides, buffer components, etc., that may negatively impact the desired downstream process or analysis. One example is the use of the ProNex® Size-Selective Purification System (Promega), a magnetic resin-based purification system for selecting double-stranded DNA.

### Compositions and Kits

Also provided by the present disclosure are compositions that include a modified TSO as described herein, including reaction mixtures. As such, the subject compositions may further include, e.g., one or more of any of the reaction mixture components described above with respect to the subject methods. For example, the compositions may further include one or more of template RNAs (e.g., mRNAs), a reverse transcriptase, dNTPs, buffers and co-factors (e.g., a salt, a metal cofactor, etc.), one or more enzyme-stabilizing components (e.g., DTT), and/or any other desired reaction mixture component(s).

In certain aspects, the subject compositions include a template ribonucleic acid (RNA) and a modified TSO of the present disclosure each hybridized to adjacent regions of a nucleic acid strand (e.g., a cDNA strand synthesized by a reverse transcriptase). The template RNA may be any template RNA of interest, e.g., an mRNA as described above.

The subject compositions may be present in any suitable environment. According to one embodiment, the composition is present in a reaction tube (e.g., a 0.2 mL tube, a 0.6 mL tube, a 1.5 mL tube, or the like) or a well. In certain aspects, the composition is present in two or more (e.g., a plurality of) reaction tubes or wells (e.g., a plate, such as a 96-well plate). The tubes and/or plates may be made of any suitable material, e.g., polypropylene, or the like. In certain aspects, the tubes and/or plates in which the composition is present provide for efficient heat transfer to the composition (e.g., when placed in a heat block, water bath, thermocycler, and/or the like), so that the temperature of the composition may be altered within a short period of time, e.g., as necessary for a particular enzymatic reaction to occur. According to certain embodiments, the composition is present in a thin-walled polypropylene tube, or a plate having thin-walled polypropylene wells. In certain embodiments it may be convenient for the reaction to take place on a solid surface or a bead. In such case, the modified TSO or one or more of the primers may be attached to the solid support or bead by methods known in the art (such as biotin linkage or covalent linkage) and reaction allowed to proceed on the support.

Other suitable environments for the subject compositions include, e.g., a microfluidic chip (e.g., a “lab-on-a-chip device”). The composition may be present in an instrument configured to bring the composition to a desired temperature, e.g., a temperature-controlled water bath, heat block, or the like. The instrument configured to bring the composition to a desired temperature may be configured to bring the composition to a series of different desired temperatures, each for a suitable period of time (e.g., the instrument may be a thermocycler).

Also included within the subject invention are kits that include one or more modified TSO as described herein. In certain embodiments, the subject kit is a cDNA library construction kit, where in certain embodiments the kit includes a modified TSO and one or more additional reagent for performing a cDNA synthesis reaction. As such, the subject kits may include, e.g., one or more of any of the reaction mixture components described above with respect to the subject methods. For example, the kits may include one or more of a template RNA (e.g., mRNAs), cDNA synthesis primer (as described herein, e.g., having a 5′ adapter region and a 3′ RNA annealing region, e.g., a poly-T sequence or a sequence complementary to at least one target RNA of interest), a reverse transcriptase, dNTPs, buffers and co-factors (e.g., a salt, a metal cofactor, etc.), one or more enzyme-stabilizing components (e.g., DTT), and/or any other desired reagents for performing a cDNA synthesis reaction. In addition, the subject kits can include reagents and/or enzymes for performing any desired downstream process or analysis, including a PCR primer pair, an amplification primer (e.g., for linear amplification), an adapter, a sequencing primer, a DNA polymerase (e.g., a thermostable polymerase for PCR), a restriction endonuclease, a ligase, or any combination thereof. Other desired kit component(s) such as containers and/or solid supports, e.g., tubes, beads, microfluidic chips, and the like, can also be included.

According to one embodiment, the subject kits include a reverse transcriptase, a cDNA synthesis primer having a 5′ adapter region and a 3′ poly-T RNA annealing region, dNTPs, and a modified TSO having a 5′ adapter region and a 3′ annealing region that includes at least one 2′-fluoro-riboguanine.

According to another embodiment, the subject kits include a reverse transcriptase, a cDNA synthesis primer having a 5′ adapter region and a 3′ poly-T RNA annealing region, dNTPs, a modified TSO having a 5′ adapter region and a 3′ annealing region that includes at least one 2′-fluoro-riboguanine, and a PCR primer pair specific for synthesis primer sites in the 5′ adapter regions of the cDNA synthesis primer and the modified TSO.

According to yet another embodiment, the subject kits include a reverse transcriptase, a cDNA synthesis primer having a 5′ adapter region and a 3′ poly-T RNA annealing region, dNTPs, a modified TSO having a 5′ adapter region and a 3′ annealing region that includes at least one 2′-fluoro-riboguanine, a PCR primer pair specific for synthesis primer sites in the 5′ adapter regions of the cDNA synthesis primer and the modified TSO, one or more adapter, and a ligase. In some of these embodiments, the adapter is a hairpin adapter, e.g., as employed in SMRT™ Sequencing (single molecule real time sequencing) from Pacific Biosciences of California, Inc. In other embodiments, the adapter is one employed for nanopore sequencing, e.g., those used for sequencing on an Oxford Nanopore sequencing platform, e.g., the MinION, PromethION, GridION, and the like.

As described in detail above, the modified TSO and/or the cDNA synthesis primer in the subject kits may include one or more useful domains/sequences in the 5′ adapter region, e.g., for use when practicing the subject methods or in any downstream application of interest. In certain aspects, the 5′ adapter region of the TSO and/or the cDNA synthesis primer can include a sequence useful for, e.g., a second-strand synthesis reaction, PCR amplification, cloning, sequencing, barcoding, molecular identification, and the like. As such, the 5′ adapter region of the TSO and/or the cDNA synthesis primer optionally comprises one or more of: a barcode sequence, a unique molecule identifier (UMI), an amplification primer sequence, a sequencing primer sequence, a nanopore sequencing adapter, a capture primer sequence (or other capture moiety), a sequence-specific nuclease cleavage site, a modified nucleotide, a biotinylated nucleotide, and a 5′ modification.

In certain embodiments, the kits include reagents for isolating RNA from a nucleic acid source. The reagents may be suitable for isolating RNA from a variety of sources including single cells, cultured cells, tissues, organs, or organisms. The subject kits may include reagents for isolating an RNA sample from a fixed cell, tissue, or organ, e.g., formalin-fixed, paraffin-embedded (FFPE) tissue. Such kits may include one or more deparaffinization agents, one or more agents suitable to de-crosslink nucleic acids, and/or the like.

Components of the subject kits may be present in separate containers, or multiple components may be present in a single container. For example, a cDNA synthesis primer and a reverse transcriptase buffer may be provided in separate containers, or may be provided in a single container. In certain embodiments, one or more kit components is provided in a lyophilized form such that the components are ready to use and may be conveniently stored at room temperature.

In addition to the above-mentioned components, a subject kit may further include instructions for using the components of the kit, e.g., to practice the subject method. The instructions for practicing the subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, Hard Disk Drive (HDD) etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are offered by way of illustration and not by way of limitation.

### Example 1: Assessment of Modified TSOs

In this Example, seven different TSOs were compared with respect to their total cDNA production and generation of full-length sequences in a SMRT™ Sequencing (single molecule real time sequencing) reaction. The seven TSOs had seven different 3′ annealing regions and identical 5′ adapter regions. The 3′ annealing regions each had three nucleotides with different modified nucleotide compositions. The sequences of the 3′ annealing regions were as follows (in a 5′ to 3′ direction): (i) rG-rG-rG; (ii) rG-rG-+G; (iii) rG-rG-fG; (iv) fG-fG-fG; (v) rG-rG-mG; (vi) rG-mG-mG; and (vii) mG-mG-mG; where rG is riboguanine (no modifications), +G is locked nucleic acid (LNA) guanine, fG is 2′-fluoro-riboguanine, and mG is 2′-O-methyl guanine (see structures in FIG. 2). The 5′ adapter region of the TSO included a forward PCR primer sequence, and the cDNA synthesis primer (the RT primer) included a 5′ adapter region with a reverse PCR primer sequence. These sequences allowed amplification using cognate forward and reverse PCR primers (described below).

Sequences of the different nucleic acid reagents are provided below in a 5′ to 3′ orientation. The RT primer and reverse PCR primer are from the NEBNext® Single Cell/Low Input cDNA Synthesis & Amplification Module (New England Biolabs Inc., Ipswich, Mass.). The TSO sequence shown below includes the non-modified annealing region (with the ribonucleotides indicated by “rG”); the modified bases in each of the modified TSOs listed above replace these three bases.

RT Primer:
(SEQ ID NO: 1)
5′-AAGCAGTGGTATCAACGCAGAGTACTTTTTTTTTTTTTTTTTTTTTT
TTTTTTTTV-3′
TSO (non-modified; rG is riboguanine):
(SEQ ID NO: 2)
5′-GCAATGAAGTCGCAGGGTTrGrGrG-3′
Reverse PCR Primer (specific for RT primer region):
(SEQ ID NO: 3)
5′-AAGCAGTGGTATCAACGCAGAGT-3′
Forward PCR Primer (specific for TSO region):
(SEQ ID NO: 4)
5′-GCAATGAAGTCGCAGGGTTG-3′

### Methods

Template switch cDNA synthesis reactions were performed with each of the TSOs listed above as follows. Certain reagents used below are commercially available from New England Biolabs (e.g., as part of an NEBNext® Single Cell/Low Input cDNA Synthesis & Amplification Module) and are indicated with “NEB”.

1. Annealing of Reverse Transcriptase (RT) Primer (Also Referred to Herein as cDNA Synthesis Primer).

For each reaction, the following components were added to a single PCR tube of a TempAs sure strip:

Reaction Mix 1

| | |
|---|---|
| Total RNA (300 ng) | ≤7 μl total |
| RT Primer Mix (NEB) | 2 μl |
| H₂O | Up to 9 μl total |
| Total Volume | 9 μl |

The tubes were mixed and incubated for 5 minutes at 70° C. and then held at 4° C. using a thermocycler.

### 2. Reverse Transcription and Template Switch Reaction

To prepare Reverse Transcription/Template Switch (RT/TS) Master Mix, components were added in the order listed at 4° C. (volumes below were multiplied by the number of reactions plus 1).

1X RT/TS Master Mix

| | |
|---|---|
| RT Buffer (NEB) | 5 μl |
| H₂O | 3 μl |
| RT Enzyme Mix (NEB) | 2 μl |
| Template Switching Oligo (12 μM) | 1 μl |
| Total Volume | 11 μl |

The RT/TS Master Mix was mixed. 11 μl of the RT/TS Master Mix was added to each tube from step 1 (each of which contained 9 μl of Reaction Mix 1) and mixed. The tubes were placed in a thermocycler at 42° C. with lid at 52° C. for 90 minutes then 70° C. with lid at 80° C. for 10 minutes.

3. cDNA Amplification by PCR.

Using the components and volumes listed below, a PCR Master Mix was made at 4° C. (volumes below were multiplied by the number of reactions plus 1). The Forward PCR Primer corresponds to a site in the TSO adapter and the Reverse PCR Primer corresponds to a site in the cDNA synthesis primer (from NEB).

1X PCR Master Mix

| PCR Master Mix (NEB) | 50 | μl |
|---|---|---|
| Forward PCR Primer (Custom) | 2 | μl |
| Reverse PCR Primer (NEB) | 2 | μl |
| 10X Cell Lysis Buffer (NEB) | 0.5 | μl |
| H₂0 | 25.5 | |
| Total Volume | 80 | μl |

To each of the RT/TS sample (20 μl total), 80 μl of the PCR Master Mix was added and mixed. The tubes were place in a thermocycler and run with the following program (lid 105° C.):

PCR program

| | | |
|---|---|---|
| 45 seconds @ 98° C. | 1 | cycle |
| 10 seconds @ 98° C. | 11 | cycles |
| 15 seconds @ 62° C. | | |
| 3 minutes @ 72° C. | | |
| 5 minutes @ 72° C. | 1 | cycle |
| Hold at 4° C. | | |

4. Purification of Amplified cDNA.

After the PCR reaction was completed, 82 μl of resuspended, room-temperature ProNex® DNA size-selection beads was added to each tube, mixed by pipetting, and incubated at room temperature for 5 minutes. The tubes were place in a magnetic stand (supplied by the manufacturer of the beads) until the supernatant was clear. The supernatant was discarded, and the beads were washed 2 times with 200 μl of freshly prepared 80% ethanol.

Once the tubes were removed from the magnetic stand, the magnetic beads were resuspended by pipette mixing in 50 μl of EB and incubated at 37° C. for 5 minutes. The tubes were placed on the magnetic stand to separate the beads from the supernatant and the supernatant was transferred to a new tube.

The amount of DNA was quantitated using a Qubit® dsDNA HS DNA quantification kit, using 1 μl of each sample. After quantitation, 1.5 ng was run on an Agilent Bioanalyzer using a High Sensitivity DNA kit (DNA was diluted to 1 ng/μl) to ensure that the amplified cDNA material had a distribution consistent with the experimental expectations.

### 5. DNA Damage Repair, End Repair, and A-Tailing.

For each sample to be processed, the following components were added to a single PCR tube of a TempAssure strip, mixed by pipetting, and spun down. Reagents indicated as being from PacBio are from the SMRTbell™ Express Template Prep Kit 2.0 provided by Pacific Biosciences of California, Inc. (Menlo Park, Calif.).

DNA Repair Reaction

| DNA Prep Buffer (PacBio) | 7 | μl |
|---|---|---|
| Purified, Amplified cDNA (80 ng to 500 ng) | ≤47.4 | μl |
| H₂O | Up to 57 μl total | |
| NAD⁺ | 0.6 | μl |
| DNA Damage Repair Mix (PacBio) | 2 | μl |
| Total Volume | 57 | μl |

These reactions were incubated at 37° C. for 30 minutes followed by a 4° C. hold in a thermocycler. With the tubes on ice, 3 μl of End Prep Mix (PacBio) was added and mixed by pipetting. The tubes were incubated at 20° C. for 30 minutes, 65° C. for 30 minutes, and held at 4° C. using a thermocycler. This step introduces a 3′ dA nucleotide that renders the ends compatible for adapter ligation in the next step.

### 6. Adaptor Ligation.

Hairpin adapters were ligated to the polished, amplified cDNA from each sample using a SMRTbell™ Express Template Prep Kit (Pacific Biosciences of California, Inc., Menlo Park, Calif.) as follows, with reagents added in the order listed.

Adapter Ligation Reaction

| | | |
|---|---|---|
| Polished DNA | 60 | μl |
| SMRT Adapter (PacBio) | 1 | μl |
| Ligation Mix (PacBio) | 30 | μl |
| Ligation Enhancer (PacBio) | 1 | μl |
| Ligation Additive (PacBio) | 1 | μl |
| Total Volume | 93 | μl |

Samples were mixed by pipetting (˜10 times) and spun to collect all liquid from the sides of the tube. The tubes were incubated at 20° C. for 60 minutes and then held at 4° C. using a thermocycler.

7. Cleanup of cDNA Libraries.

After the ligation reaction was completed, 93 μl of resuspended, room-temperature ProNex® DNA size-selection beads was added to each tube, mixed by pipetting (˜10 times), and incubated at room temperature for 5 minutes. The tubes were place in a magnetic stand (supplied by the manufacturer of the beads) until the supernatant was clear. The supernatant was discarded and the beads were washed 2 times with 200 μl of freshly prepared 80% ethanol.

Once the tubes were removed from the magnetic stand, the magnetic beads were resuspended by pipette mixing in 20 μl of EB and incubated at 37° C. for 5 minutes. The tubes were placed on the magnetic stand to separate the beads from the supernatant and the supernatant was transferred to a new tube.

The amount of DNA was quantitated using a Qubit® dsDNA HS DNA quantification kit, using 1 μl of each sample. After quantitation, 1.5 ng was run on an Agilent Bioanalyzer using a High Sensitivity DNA kit (DNA was diluted to 1 ng/μl) to estimate library size for iTube formation and diffusion loading onto a SMRT™ Cell 1M sequencing chip (Pacific Biosciences of California, Inc., Menlo Park, Calif.). SMRT™ Sequencing (single molecule real time sequencing) was performed on a Sequel™ sequencing instrument according to manufacturer's instructions.

### Results

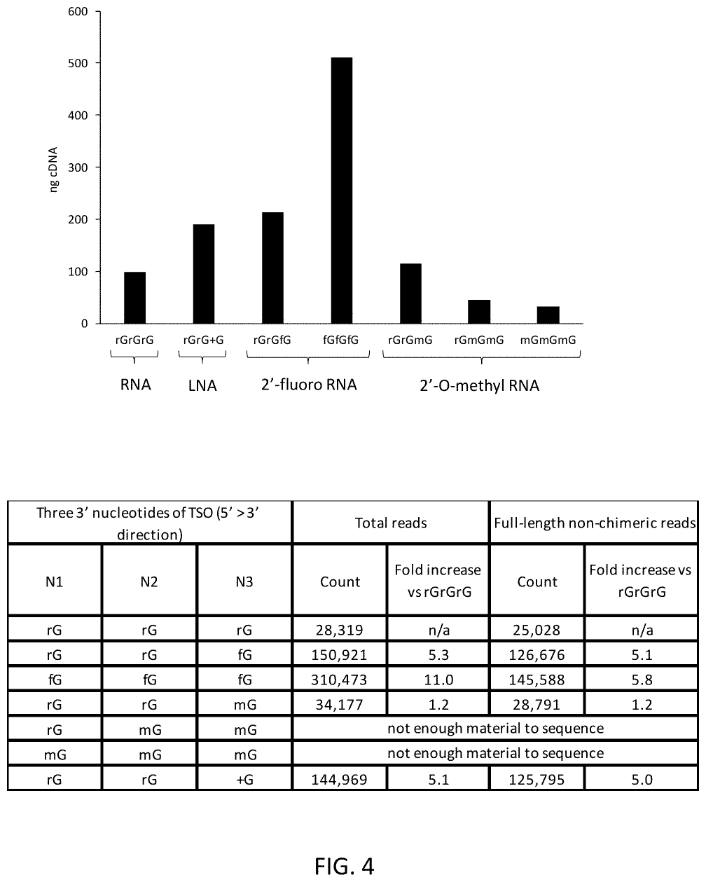

FIG. 4, top panel, shows the total amount of cDNA generated using each of the different TSOs. The TSO with unmodified rG ribonucleotides (rGrGrG) generated approximately 100 ng of cDNA. TSOs with 3′ annealing regions that contain 2′-fluoro-ribiguanine nucleotides show increased cDNA production as compared to the rGrGrG TSO, with 3′ annealing region rGrGfG (one 2′-fluoro-riboguanine) increasing the amount of cDNA produced approximately by a factor of two (˜200 ng) and 3′ annealing region fGfGfG (three 2′-fluoro-riboguanines) increasing the amount of cDNA approximately by a factor of five (˜500 ng). In contrast, TSOs with 3′ annealing regions containing 2′-O-methyl-riboguanine residues either had no effect as compared to the rGrGrG TSO (3′ TSO with a single 2′-O-methylguanine; rGrGmG) or reduced the amount of cDNA produced as compared to the rGrGrG TSO (3′ TSOs with either one or two 2′-O-methylguanine residues; rGmGmG and mGmGmG). TSOs with a single LNA nucleotide (rGrG+G) also showed increased cDNA production.

FIG. 4, bottom panel shows the results of sequence analysis of the cDNAs produced in the top panel. Total reads and full length non-chimeric reads (FLNC reads) are shown.

While the fGfGfG TSO produced significantly more product, there was a significant amount of incomplete and/or chimeric cDNA products (non-FLNC reads) that are not useful for transcriptome analysis (see bottom panel of FIG. 4). The predominant non-FLNC reads we encountered include those that have the same primer sequence at each end (e.g., cDNA synthesis primer sequences at both ends or TSO sequences at boteh ends) and those in which the TSO annealed at a site within the mRNA template rather than the at the 3′ overhang nucleotides at the 5′ end of the mRNA. We tried two approaches to reduce these off-target products: temporally separating the RT and TSO reactions (Example 2) and performing a cDNA clean-up step prior to cDNA amplification (Example 3).

### Example 2: Temporal Separation of RT and TSO Addition

We performed experiments in which the timing of TSO addition to the cDNA synthesis reaction was varied. Without limitation to any particular mechanism, we hypothesized that allowing first strand cDNA synthesis to complete before TSO addition would favor annealing of the TSO to the 3′ overhang region rather than to internal mRNA sites.

The following modified TSOs were analyzed: (i) rG-rG-+G (LNA); (ii) rG-rG-fG (rrF); (iii) fG-fG-fG (FFF). For each of these modified TSOs, separate cDNA synthesis reactions were performed in which the modified TSO was added either at the start of the cDNA synthesis reaction (0 min.; i.e., identical to the reactions described in Example 1) or 30 min., 45 min., 60 min., or 75 min. after the cDNA synthesis reaction had started. These reactions were processed to determine the amount of cDNA generated and/or for the percentage of full length non-chimeric (FLNC) reads generated after performing subsequent steps in the process (as described above).

### Results

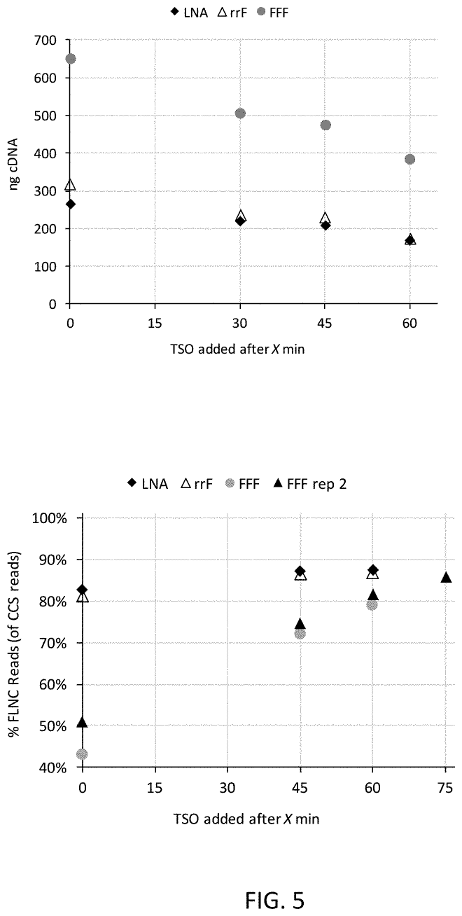

The top panel of FIG. 5 shows total cDNA production (in nanograms; ng) from the 0, 30, 45, and 60 minute timepoints. As seen in FIG. 4, the cDNA synthesis reactions that used the FFF modified TSO generated significantly more cDNA than the reactions that used the LNA or rrF TSOs at all timepoints (generally at least 2-fold more). In addition, for all three modified TSOs used, the amount of cDNA produced was reduced as the timing of TSO addition increased.

The bottom panel of FIG. 5 shows the percentage of FLNC reads obtained from 0, 45, and 60 minute timepoints for LNA, rrF, and duplicate FFF reactions as well as a 75 minute timepoint for one of the FFF reactions. As shown, the percent of FLNC reads increased only slightly for the LNA and rrF TSOs (from approximately 80% to approximately 87%). However, the percent FLNC reads was dramatically increased for both of the duplicate FFF TSO reactions as the time of modified TSO addition was increased. Specifically, the percent FLNC reads increased from approximately 43% FLNC reads at time 0 to approximately 80% FLNC reads at the 60 minute timepoint and 85% FLNC reads at the 75 minute time point. Given that the FFF TSO produced significantly more total cDNA than the LNA or rrF, the total number of FLNC reads was estimated to be significantly higher at these later time points.

### Example 3: cDNA Clean-Up Prior to cDNA Amplification

We next performed an experiment comparing total cDNA yield and FLNC reads in the standard assay (as described above) and assays in which the cDNA sample was cleaned up prior to the PCR amplification step. Without limitation to any particular mechanism, this step is expected to reduce the production of non-FLNC reads by removing the cDNA synthesis and modified TSO primers from the PCR reaction which could contribute to background amplification products. In addition to the clean-up step, reactions were performed with increasing concentrations of the modified TSO (0.6 μM, 2.4 μM, 0.6 μM). Experiments comparing the impact of the clean-up step on the rG-rG-fG TSO were performed at the 0.6 μM concentration while those done with the rG-fG-fG TSO were performed at all three concentrations.

After the RT/TS reactions were completed (as described in Example 1), 30 μl of Elution Buffer (EB; 10 mM Tris-Cl, pH 8.5) was added to each reaction followed by the addition of 52 μl of ProNex® beads (a magnetic resin from Promega employed for nucleic acid size selection). The reactions were gently pipette mixed and incubated at room temperature for 5 minutes. The tubes were place in a magnetic stand (supplied by Promega) until the supernatant was clear. The supernatant was discarded and the beads were washed 2 times with 200 μl of freshly prepared 80% ethanol.

Once the tubes were removed from the magnetic stand, the magnetic beads were resuspended by pipette mixing in 46 μl of EB, quick spun to collect all liquid from the sides of the tubes, and incubated at 37° C. for 5 minutes. The tubes were placed on the magnetic stand to separate the beads from the supernatant and 45.5 μl of the supernatant was transferred to a new tube. These supernatants were processed as described in steps 3-10 in Example 1 (PCR amplification through sequence analysis).

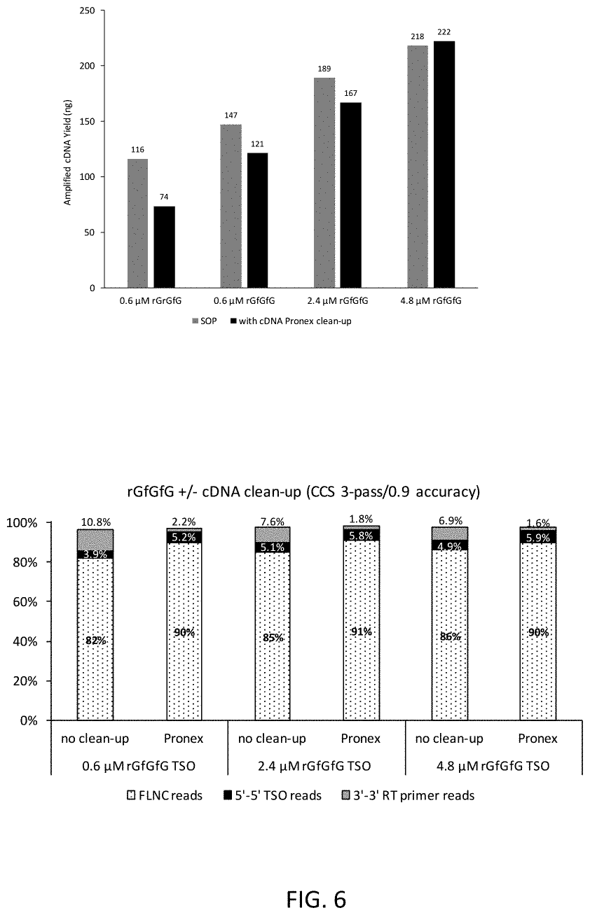

The top panel of FIG. 6 shows the cDNA yield in nanograms (ng) from all reactions performed both without (standard operating procedure; SOP) and with cDNA bead clean-up. At the 0.6 mM and 2.4 mM TSO concentrations, the SOP resulted in increased amounts of cDNA as compared to the cDNA bead clean-up samples. At 4.8 mM concentration, the cDNA levels were approximately equivalent. In addition, the total cDNA yield was higher using the rG-fG-fG TSO as compared to the rG-rG-fG TSO at the lowest concentration. Moreover, the total yield was increased for the rG-fG-fG TSO as its concentration was increased in the RT/TS reaction.

The bottom panel of FIG. 6 shows the percentage of FLNC, 5′-5′ TSO, and 3′-3′ RT primer reads (the latter two representing undesired products) for the rGfGfG TSO samples. For all TSO concentrations, the clean-up samples showed significantly higher FLNC reads than the non-clean-up reads, ranging from 6-8% increased yield.

In view of this data, increasing the concentration of the modified TSO results in increased cDNA yield while performing a clean-up step after the RT/TS reaction leads to increased percentage of FLNC product (the desired product).

### CONCLUSION

In view of the data presented in the Examples above, modified TSOs that include at least one 2′fluoro-riboncleotide result in improved cDNA yield. In addition to using such modified TSOs, delaying the timing of TSO addition to the cDNA synthesis reaction (i.e., at a time after cDNA synthesis has been initiated), cleaning up the cDNA sample prior to subsequent amplification reactions, and increasing the concentration of the modified TSO can also improve the yield of desired cDNA products (e.g., FLNC cDNAs) in these reactions. These findings allow for the implementation of improved analysis of samples with low amounts of RNA, e.g., low or single cell samples, samples with dilute RNA species (e.g., circulating RNA), and the like.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A method for generating a complementary DNA (cDNA) strand with a 3′ adapter region, the method comprising:
combining an RNA template with a cDNA synthesis primer, a template switching oligonucleotide (TSO), and a reverse transcriptase under cDNA synthesis conditions, wherein the TSO comprises a 5′ adapter region and a 3′ annealing region comprising at least one 2′-fluoro-ribonucleotide, wherein:
(i) the cDNA synthesis primer anneals to the RNA template and the reverse transcriptase generates an RNA-cDNA intermediate from the annealed cDNA synthesis primer, wherein the cDNA strand of the RNA-cDNA intermediate comprises a 3′ overhang; and
(ii) the 3′ annealing region of the TSO anneals to the 3′ overhang of the RNA-cDNA intermediate and the reverse transcriptase extends the 3′ end of the cDNA strand of the RNA-cDNA intermediate using the annealed TSO as a template;
thereby generating a cDNA strand comprising a 3′ adapter region.

2. The method of claim 1, wherein the 3′ annealing region comprises three ribonucleotide residues.

3. (canceled)

4. The method of claim 2, wherein the 3′ annealing region comprises two 2′-fluoro-ribonucleotides.

5. (canceled)

6. The method of claim 1, wherein the at least one 2′-fluoro-ribonucleotide is 2′-fluoro-riboguanine (2′fG).

7-10. (canceled)

11. The method of claim 1, wherein the 3′ annealing region of the TSO, in a 5′ to 3′ direction, is selected from the group consisting of: rG-rG-2′fG; rG-2′fG-2′fG; 2′fG-2′fG-2′fG; 2′fG-2′fG-2′fG-2′fG; rN-2′fG-2′fG; rI-2′fG-2′fG; and 5′NI-2′fG-2′fG.

12. The method of claim 11, wherein the 3′ annealing region of the TSO, in a 5′ to 3′ direction, is: rG-2′fG-2′fG.

13. The method of claim 11, wherein the 3′ annealing region of the TSO, in a 5′ to 3′ direction, is: 2′fG-2′fG-2′fG.

14. The method of claim 1, further comprising amplifying the cDNA strand comprising the 3′ adapter region.

15. The method of claim 1, wherein the 5′ adapter region of the TSO further comprises one or more of: a barcode sequence, a unique molecule identifier (UMI), an amplification primer sequence, a sequencing primer sequence, a capture primer sequence, a sequence-specific nuclease cleavage site, a modified nucleotide, a biotinylated nucleotide, and a 5′ modification.

16-20. (canceled)

21. The method of claim 1, wherein the RNA template is selected from the group consisting of: mRNA, non-coding RNA, miRNA, siRNA, piRNA, lncRNA, and ribosomal RNA.

22. The method of claim 21, wherein the RNA template is an mRNA.

23. (canceled)

24. The method of claim 22, wherein the mRNA template has a poly-A tail at the 3′-end, and wherein the cDNA synthesis primer comprises a 3′ poly-T sequence complementary to the poly-A tail.

25-27. (canceled)

28. The method of claim 1, wherein the cDNA synthesis primer and the reverse transcriptase are combined with the RNA template under cDNA synthesis conditions to form a pre-extension mixture to generate the RNA-cDNA intermediate prior to combining with the TSO.

29. The method of claim 28, wherein the pre-extension mixture is incubated from 10 minutes to 4 hours prior to combining with the TSO.

30. The method of claim 29, wherein the pre-extension mixture is incubated from 30 minutes to 2 hours prior to combining with the TSO.

31. (canceled)

32. A method for generating adapter-containing cDNAs from a sample comprising mRNAs, the method comprising:
(a) obtaining a sample comprising mRNAs having 3′ poly-A tails;
(b) producing a cDNA synthesis reaction by contacting the sample with a cDNA synthesis primer and a reverse transcriptase under cDNA synthesis conditions, wherein the cDNA synthesis primer comprises a 3′ poly-T annealing region and the reverse transcriptase adds 3′ terminal nucleotide overhangs to the 3′ ends of cDNAs;
(c) allowing the cDNA synthesis reaction to proceed for from 10 minutes to 4 hours to produce cDNAs with 3′ overhangs;
(d) adding a template switching oligonucleotide (TSO) to the cDNA synthesis reaction, wherein the TSO comprises a 5′ adapter region and a 3′ annealing region comprising three ribonucleotides, wherein at least one of the ribonucleotides is a 2′-fluoro-riboguanine (2′fG) nucleotide; and
(e) incubating the cDNA synthesis reaction under conditions that allow the 3′ annealing region of the TSO to anneal to the 3′ overhangs of the cDNAs and that allow extension of the 3′ end of the cDNAs using the annealed TSO as a template, thereby generating adapter-containing cDNAs.

33-35. (canceled)

36. The method of claim 32, wherein any non-2′fG nucleotides in the 3′ annealing region of the TSO are riboguanine (rG) nucleotides.

37. The method of claim 32, wherein the 3′ annealing region of the TSO, in a 5′ to 3′ direction, is selected from the group consisting of: rG-rG-2′fG; rG-2′fG-2′fG; 2′fG-2′fG-2′fG; 2′fG-2′fG-2′fG-2′fG; rN-2′fG-2′fG; rI-2′fG-2′fG; and 5′NI-2′fG-2′fG.

38. The method of claim 37, wherein the 3′ annealing region of the TSO, in a 5′ to 3′ direction, is: rG-2′fG-2′fG.

39. The method of claim 37, wherein the 3′ annealing region of the TSO, in a 5′ to 3′ direction, is: 2′fG-2′fG-2′fG.

40-63. (canceled)


SEQUENCE LISTING (4 sequences)

SEQ ID NO: 1 - DNA, 56 bp, Artificial Sequence
  synthetic oligonucleotide primer
aagcagtggtatcaacgcagagtacttttttttttttttttttttttttttttttv

SEQ ID NO: 2 - DNA, 22 bp, Artificial Sequence
  synthetic template switching oligonucleotide
  misc_feature (20)..(22): riboG
gcaatgaagtcgcagggttggg

SEQ ID NO: 3 - DNA, 23 bp, Artificial Sequence
  synthetic oligonucleotide primer
aagcagtggtatcaacgcagagt

SEQ ID NO: 4 - DNA, 20 bp, Artificial Sequence
  synthetic oligonucleotide primer
gcaatgaagtcgcagggttg